(19) United States
(12) Patent Application Publication
(10) Pub. No.: US 2019/0214127 A1
(43) Pub. Date: Jul. 11, 2019

(54) SUB-OPTIMAL HEALTH DETECTION AND ALERT GENERATION USING A TIME SERIES OF IMAGES

(71) Applicant: International Business Machines Corporation, Armonk, NY (US)

(72) Inventors: KUN HU, Santa Clara, CA (US); James Kaufman, San Jose, CA (US); MIN LI, San Jose, CA (US); Valentina Salapura, YORKTOWN HEIGHTS, NY (US)

(73) Assignee: International Business Machines Corporation, Armonk, NY (US)

(21) Appl. No.: 15/867,216

(22) Filed: Jan. 10, 2018

(51) Int. Cl. G16H 30/40; G16H 50/20; G06T 7/00; G06T 5/00; G06T 5/20; A61B 1/24; A61B 3/14; A61B 5/00; A61B 5/103

(52) U.S. Cl. CPC G16H 30/40; G16H 50/20; G06T 7/0016; G06T 5/002; G06T 5/20; A61B 1/24; G06T 2207/30041; A61B 5/0077; A61B 5/1032; A61B 5/7282; G06T 2207/30088; G06T 2207/30201; G06T 2207/30036; A61B 3/14

(57) ABSTRACT

A series of images of a body part of a user is received. The series of images of the body part of the user are captured over a predetermined time period. A feature of the body part is extracted from the series of images, in which a change in appearance of the extracted feature over the predetermined time period is indicative of a potential health condition of the user. The extracted feature is compared to one or more reference images from a knowledge base to determine a difference between the extracted feature and the one or more reference images. The one or more reference images correlate a particular body feature to a particular potential health condition. A potential health condition of the user is detected based upon the comparing. A notification is sent based upon the detected potential health condition.

### TECHNICAL FIELD

The present invention relates generally to a method, system, and computer program product for detecting health status of a user. More particularly, the present invention relates to a method, system, and computer program product for sub-optimal health detection and alert generation using a time series of images of a user.

### BACKGROUND

It is often too late when people detect symptoms that lead to degraded health conditions including the presence of severe diseases such as melanoma or other cancer. Many dangerous diseases and other health degradation processes occur over a period of time. Many of these conditions are noticeable on the surface of the skin (e.g., of the face or the body) and other external facial or other appearance characteristics. Examples of such appearance characteristics include changes in eye size or the joints of fingers. Since the processes leading to change in external are often gradual, changes from day to day in facial or other appearance characteristics are not easily noticeable to a naked human eye.

### SUMMARY

The illustrative embodiments provide a method, system, and computer program product. An embodiment of a method includes receiving a series of images of a body part of a user, the series of images of the body part of the user captured over a predetermined time period. The embodiment further includes extracting a feature of the body part from the series of images, in which a change in appearance of the extracted feature over the predetermined time period is indicative of a potential health condition of the user. The embodiment further includes comparing the extracted feature to one or more reference images from a knowledge base to determine a difference between the extracted feature and the one or more reference images, the one or more reference images correlating a particular body feature to a particular potential health condition. The embodiment further includes detecting a potential health condition of the user based upon the comparing, and sending a notification based upon the detected potential health condition.

In an embodiment, detecting the potential health condition further includes determining that the difference exceeds a threshold. An embodiment further includes applying image correction to at least one of the series of images to filter out noise due to one or more environmental factors present during capturing of the at least one image.

In an embodiment, the notification is sent to at least one of the user and a medical provider associated with the user. An embodiment further includes determining that the potential health condition is a potentially acute health condition. In an embodiment, the notification is an emergency alert indicating the potentially acute health condition.

An embodiment further includes receiving activity information from a wearable device associated with the user, the activity information indicative of activity of the user, and determining a reason for the detected potential health condition based upon the activity information.

In an embodiment, the body part comprises at least one of a face of the user, a hand of the user, a torso of the user, a leg of the user, a neck of the user, and a skin of the user.

In another embodiment, the feature includes at least one of a facial anomaly, a skin condition of the body part, an eye condition of the user, a teeth condition of the user, a change in shape of the body part, and a color of the body part.

An embodiment includes a computer usable program product. The computer usable program product includes one or more computer-readable storage devices, and program instructions stored on at least one of the one or more storage devices.

An embodiment includes a computer system. The computer system includes one or more processors, one or more computer-readable memories, and one or more computer-readable storage devices, and program instructions stored on at least one of the one or more storage devices for execution by at least one of the one or more processors via at least one of the one or more memories.

### BRIEF DESCRIPTION OF THE DRAWINGS

Certain novel features believed characteristic of the invention are set forth in the appended claims. The invention itself, however, as well as a preferred mode of use, further objectives and advantages thereof, will best be understood by reference to the following detailed description of the illustrative embodiments when read in conjunction with the accompanying drawings, wherein:

FIG. 1 depicts a block diagram of a network of data processing systems in which illustrative embodiments may be implemented;

FIG. 2 depicts a block diagram of a data processing system in which illustrative embodiments may be implemented;

FIG. 3 depicts a block diagram of an example configuration for sub-optimal health detection and alert generation using a time series of images of a user in accordance with an illustrative embodiment;

FIG. 4 depicts example facial characteristics in accordance with an illustrative embodiment;

FIG. 5 depicts a flowchart of an example process for sub-optimal health detection and alert generation using a time series of images of a user in accordance with an illustrative embodiment;

FIG. 6 depicts a flowchart of another example process for sub-optimal health detection and alert generation using a time series of images of a user in accordance with an illustrative embodiment; and

FIG. 7 depicts a flowchart of another example process for sub-optimal health detection and alert generation using a time series of images of a user in accordance with an illustrative embodiment.

### DETAILED DESCRIPTION

The illustrative embodiments used to describe the invention generally address and solve the above-described problems and other problems related to sub-optimal health detection of a user. With devices such as smart phones, taking photos of oneself (i.e., selfies) can be very easy. In addition, cameras are available at a low cost and are easily accessible for such utility. Accordingly, a user can capture a series of images of the user over a period of time.

One or more embodiments are directed to a method and system for sub-optimal health detection and alert generation using a time series of images of a user. In accordance with various embodiments, a series of images of a user are captured over a period of time by a smartphone or other device having a camera and sent to a server. In an embodiment, the server analyzes the images to identify appearance characteristics, such as facial features, that may be indicative of a health status of the user. In a particular embodiment, facial features are correlated with a health status of a particular internal organ or skin surface of the user.

In one or more embodiments, the server detects abnormal changes in a particular body feature that may be indicative that a person is in a sub-optimal health state. In one or more embodiments, an application facilitates detection of a deterioration in health condition through image recognition and comparison with a standard image and alerts a user with a notification to schedule a physical checkup with a medical professional for confirmation of the condition and/or instructions to improve the user's living style for health improvement. One advantage offered by at least one embodiment is to dramatically reduce personal medical cost due to earlier detection of potentially costly medical conditions.

In an embodiment, a client device, such as a smartphone, having an embedded camera captures a series of images of a user over a period of time and sends the images to a server. In the embodiment, the server analyzes the series of images (e.g., time series data on features detected from each image of a same client over time) and detects a potential medical problem or other health status or condition based upon changes in one or more facial or other body features in the images. Upon detecting a potential medical problem, the server triggers an alert that is sent to the user with a recommendation to address the potential problem by, for example, scheduling a physician visit to check/confirm the user's health status.

In a particular embodiment, the client device is configured to remind the user to take a new image if a new image is due and the user has not yet taken the new image. In another particular embodiment, the server is further configured to identify a positive status change, for example, that an eczema is healing, and communicate the positive status change to the user in order to reinforce positive behavior. It should be understood that in various embodiments that the procedures described herein are not meant to replace diagnostic by a medical professional, but to provides possible early warning for further medical consultation and voluntarily changes of living style that cannot be easily identified by the naked eye. The system also identifies the status

An embodiment includes a system architecture which is composed of multiple components including a client device having an associated camera module, a server, and one or more databases in communication via a network. In particular embodiments, the client device includes a mobile phone or a camera or other imaging device installed at a user location. In the embodiment, the client device captures images of a user periodically (e.g., at the same time/condition every day) and/or on demand and sends the images to the server and the one or more databases. In particular embodiments, the one or more databases store the time series of images as well as health information regarding the user's health information. In particular embodiments, the user's health information includes quantitative measurements for determining differences between time-series image data (e.g., “selfies” taken over a period of time). In particular embodiments, the image database can also be augmented by accessing images of the user available on the Internet, such as from a social media site, if necessary. In some embodiments, one or more of the databases may be implemented in a network cloud.

In the embodiment, the server includes a facial and body recognition module configured to recognize a user's face or other body portion from the series of images and extract features that are related to health status of the user. In the embodiment, the server further includes a comparison module configured to compare the series of images against a series of photos taken across an adjustable time window to identify changes in one or more features extracted from the time-series of images. In the embodiment, the server further includes a sub-health detector module configured to receive input from comparison module and a knowledge of mapping between facial and body changes and internal health or skin diseases and other health conditions, and detect any anomalies indicated by the analysis of the time series of images. In the embodiment, the server further includes an alert/notification module configured to create alerts regarding a potentially dangerous health condition, or communicate any long-term or short-term health status change to the user or other entity such as a physician. Although various embodiments describe the recognition, comparison, and sub-health detection procedures being formed by the server, it should be understood that in other embodiments one or more of such procedures may be formed by the client device alone or in combination with the server.

In a particular embodiment, the images of the user are preferably taken at the approximately same time of the day, same place, and same condition (e.g., preferably with no makeup applied to the user's face). In particular examples, the image can be captured at morning time while performing a morning routine, for example in a bathroom after brushing one's teeth.

In some embodiments, the client device and/or server is configured to process the time series of images to filter out factors that are due to changes from an external environment instead of the user himself/herself such as filtering out skin color changes due to lighting conditions. In other embodiments, if the environment of taking a current image is dramatically different one from a previous tracking record, the system can detect and alert the user for further action such as either re-taking the current image, or discarding the current image if a retake is not possible in order to avoid confusion and/or an inaccurate suggestion.

In one or more embodiments, the system allows the user to input information related to a current condition such as lighting, weather (e.g, a sunny or a cloudy day), and whether the user is wearing makeup or not to further facilitate adjustment of the image comparison algorithm. In various embodiments, capturing an image on an individual day could be skipped and if too many days are skipped in a row, the system generates an alert to the user such as creating a notification on a mobile phone, sending an email, sending a text message or some other preferred methods of contact that includes an indication to a user that an image should be taken.

In a particular embodiment, a smart phone is used as the client device for taking daily photos of a user. In another particular embodiment some other smart device is used to take photos such as a smart watch. In yet another particular embodiment, a fixed camera installed in a room such as a bathroom is used to capture images of a user.

In an embodiment, the system retains taken images of a user, analyzes the images by image recognition, and tags changes in areas of features identified from one image in a sequence image in the series as potential problem area. In the embodiment, the tagged area is checked in each new image to determine if the problem increases or worsens. If over time any problem area reaches a threshold level, the system (e.g., the server) generates an alert. In particular embodiments, the system is configured to instruct users to take an image of a particular part of body to have a closer and more precise monitoring. For example, if dark circles are developing under the eyes of the user, the system instructs the user to take detailed photos of the eyes on a regular basis with greater resolution to improve the accuracy of the image comparison and the recommendation. In one or more embodiments. The threshold is not predetermined for all clients but is instead calibrated and varied for each client using images taken by the client over time.

In particular embodiments, a system database or cloud storage system records possible problems which can be noticed on the skin, the face or the body of a user. In a particular example, the system keeps records of various images of melanoma progression over time, such as dark or red spots on the skin, and compares a knowledge base including an image database for certain health issues available and provided by a medical agency with the user's daily images to provide further information to the user about the user's health status.

In particular embodiments, the system detects certain changes on the face and body which indicate a potential health problem. An examples of such a change includes dark circles under the eyes over a period of time which could indicate kidney problem as opposed to circles caused by insufficient sleep. Another example includes detecting an increase of how much a user's eyes are protruding. A change in how much a user's eyes are bulging out of the normal position could potentially indicate potential thyroid gland hyperactivity. In another example, some red spots detected on the skin might be an early symptom for a skin cancer if not treated early enough. In another example, a detected enlargement of joints of fingers or legs of a user may indicate a health issue an individual may have which can be detected by comparing the images over time. In terms of changes of other parts of the body, such as hands, legs, neck, torso, back, another example is a detection of an early hunchback condition because of an incorrect posture when using computer. In other examples, the system detects swelling, skin acnes, color changes, skin burn of the body, etc. In other particular example, the system tracks the development of an injury, wound that is healing, skin condition, or other health condition on various parts of the body to determine through the time progression of images if the status is improving, deteriorating, or remains the same.

In one embodiment, a user's health profile stored in the database is used to provide information regarding the user's family medical history and personal medical history to determine if the person has predisposition to certain conditions. For example, history of family thyroid problems would be indicative to particularly track appearance of eyes for the users. In another embodiment, the system receives health monitoring information from a user's health monitoring devices such as a smart watch or fitness tracker device to detect changes of life style of the user and learn a correlation between detected facial and body changes and changes of living style or habit.

In one or more embodiments, the system is configured to differentiate acute and chronic disease and reacts differently. When slow changes that may indicate a progressive disease exceed a threshold, the system can trigger alerts calling for closer attention. On the other hand, when an emergency condition or event is detected, the system generates an alert with higher priority and suggests a clinic visit or other consultation with a medical professional as soon as possible.

In another embodiment, the system detects emergency and generates a higher priority alert to the user. In an example scenario, a user has appendicitis and the system detects dark rings around his/her eyes from the internal infection. The system recognizes the emergency condition and generates an alert instructing the user to go to an emergency room immediately.

In an embodiment, a system tracks a history of user images taken over a period of time, conducts facial recognition to compare historical images to characteristics of a healthy face or from a medical image database for a specific health concern, detects a health concern based upon the comparison, and alerts the user based upon the detected health concern. In particular embodiments, tracked facial characteristics include, but are not limited to, dark circles around the eyes, eyes that bulge out, dull complexion, unhealthy color of teeth, dark spots on the skin, skin inflammations, change of shape of face, changes in eyeball shape/color, and acne. In another particular embodiment, the system acquires information from wearable devices to analyze the user's activities to facilitate determining a reason for a detected health concern.

In an embodiment, the system performs preprocessing to get rid images of noise data that affects the accuracy of the analysis such as external environmental factors. In a particular embodiment, the system differentiates users with makeup from users without makeup or detects the skill color changes due to lighting conditions. In other particular embodiments, the system allow a user to manually correct the external environmental factors if possible by allowing the user to indicate one or more external environmental factors present during capturing of a particular image.

In particular embodiments, the system considers other anomalies in addition to instead of facial anomalies such as anomalies in other parts of the body such as hands, legs, torso, neck, back in terms of skin acnes, color changes, skin burn, changes of body shape indicating body swelling or abnormal weight changes.

In particular embodiments, if anomalies are detected, the system creates alerts and provide suggestions to the user to mitigate the anomalies such as changing towards healthier life styles and eating habits. In other particular embodiments, the system connects with a user's calendar to detect whether the user is under pressure and working too hard which might lead to sub-healthy status of the user. In another particular embodiment, the system acquires information from a personal health tracking system (e.g., a smart watch or fitness tracker) with the user's permission to reevaluate activity goals if some anomalies are detected.

In particular embodiments, the system analyzes a time series of images of a user captured over a period of months or years to detect progressive changes over the period of time which might indicate long term health degradation, and create alerts to inform the user of the detected progressive change. In other particular embodiments, the system is configured to detect sudden changes to the appearance of the user over a period of days of weeks which might indicate infection, infectious disease, toxin exposure, food poisoning, or other rapid onset adverse health conditions, and create alerts to the user informing the user of the detected sudden changes.

The manner of sub-optimal health detection and alert generation using a time series of images of a user is unavailable in the presently available methods. A method of an embodiment described herein, when implemented to execute on a device or data processing system, comprises substantial advancement of the functionality of that device or data processing system in providing sub-optimal health detection and alert generation.

An embodiment can be implemented as a software application. The application implementing an embodiment can be configured as a modification of an existing health status detection system, as a separate application that operates in conjunction with an existing health status detection system, a standalone application, or some combination thereof.

The illustrative embodiments are described with respect to certain types of health detection and alert generation procedures and algorithms, devices, data processing systems, environments, components, and applications only as examples. Any specific manifestations of these and other similar artifacts are not intended to be limiting to the invention. Any suitable manifestation of these and other similar artifacts can be selected within the scope of the illustrative embodiments.

Furthermore, the illustrative embodiments may be implemented with respect to any type of data, data source, or access to a data source over a data network. Any type of data storage device may provide the data to an embodiment of the invention, either locally at a data processing system or over a data network, within the scope of the invention. Where an embodiment is described using a mobile device, any type of data storage device suitable for use with the mobile device may provide the data to such embodiment, either locally at the mobile device or over a data network, within the scope of the illustrative embodiments.

The illustrative embodiments are described using specific code, designs, architectures, protocols, layouts, schematics, and tools only as examples and are not limiting to the illustrative embodiments. Furthermore, the illustrative embodiments are described in some instances using particular software, tools, and data processing environments only as an example for the clarity of the description. The illustrative embodiments may be used in conjunction with other comparable or similarly purposed structures, systems, applications, or architectures. For example, other comparable mobile devices, structures, systems, applications, or architectures therefor, may be used in conjunction with such embodiment of the invention within the scope of the invention. An illustrative embodiment may be implemented in hardware, software, or a combination thereof.

The examples in this disclosure are used only for the clarity of the description and are not limiting to the illustrative embodiments. Additional data, operations, actions, tasks, activities, and manipulations will be conceivable from this disclosure and the same are contemplated within the scope of the illustrative embodiments.

Any advantages listed herein are only examples and are not intended to be limiting to the illustrative embodiments. Additional or different advantages may be realized by specific illustrative embodiments. Furthermore, a particular illustrative embodiment may have some, all, or none of the advantages listed above.

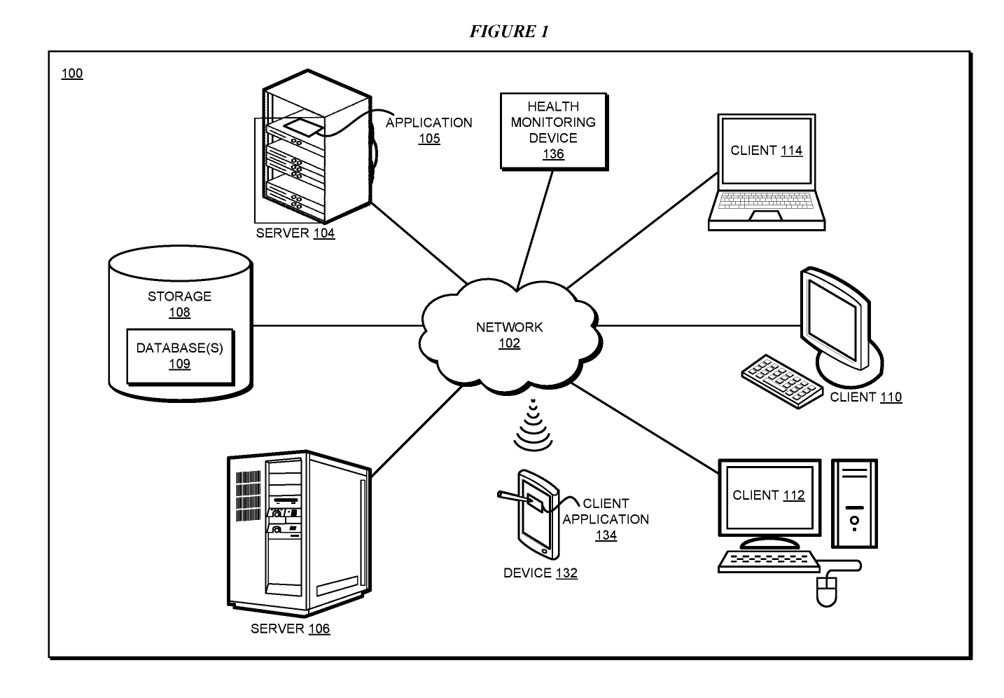

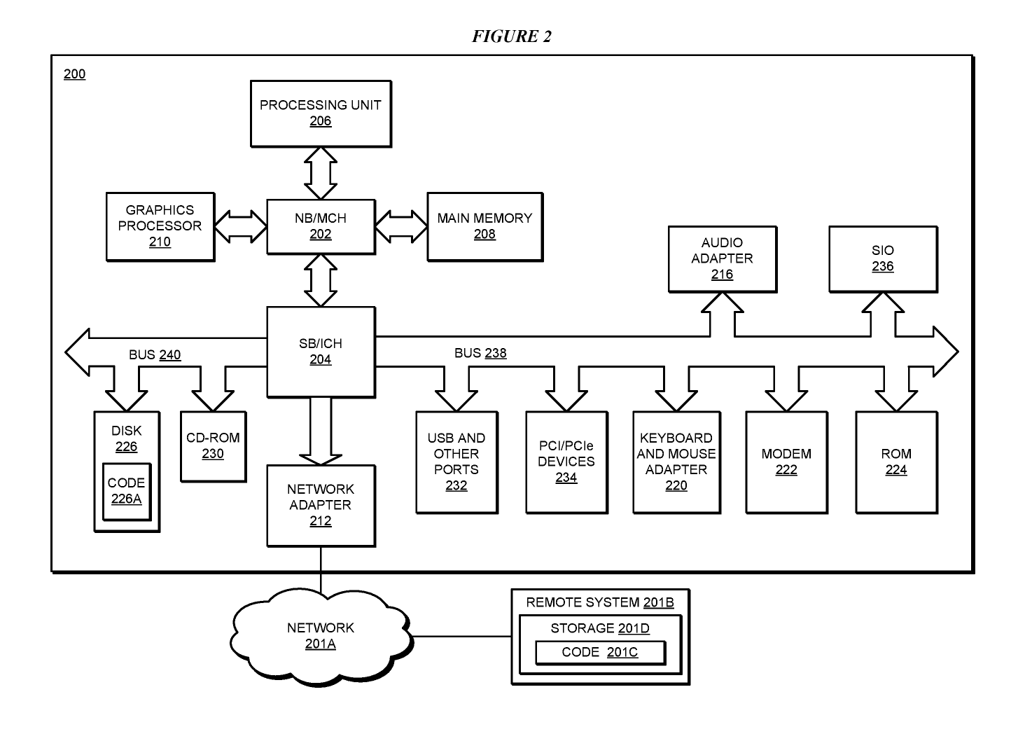

With reference to the figures and in particular with reference to FIGS. 1 and 2, these figures are example diagrams of data processing environments in which illustrative embodiments may be implemented. FIGS. 1 and 2 are only examples and are not intended to assert or imply any limitation with regard to the environments in which different embodiments may be implemented. A particular implementation may make many modifications to the depicted environments based on the following description.

FIG. 1 depicts a block diagram of a network of data processing systems in which illustrative embodiments may be implemented. Data processing environment 100 is a network of computers in which the illustrative embodiments may be implemented. Data processing environment 100 includes network 102. Network 102 is the medium used to provide communications links between various devices and computers connected together within data processing environment 100. Network 102 may include connections, such as wire, wireless communication links, or fiber optic cables.

Clients or servers are only example roles of certain data processing systems connected to network 102 and are not intended to exclude other configurations or roles for these data processing systems. Server 104 and server 106 couple to network 102 along with storage unit 108. Software applications may execute on any computer in data processing environment 100. Clients 110, 112, and 114 are also coupled to network 102. A data processing system, such as server 104 or 106, or client 110, 112, or 114 may contain data and may have software applications or software tools executing thereon.

Only as an example, and without implying any limitation to such architecture, FIG. 1 depicts certain components that are usable in an example implementation of an embodiment. For example, servers 104 and 106, and clients 110, 112, 114, are depicted as servers and clients only as example and not to imply a limitation to a client-server architecture. As another example, an embodiment can be distributed across several data processing systems and a data network as shown, whereas another embodiment can be implemented on a single data processing system within the scope of the illustrative embodiments. Data processing systems 104, 106, 110, 112, and 114 also represent example nodes in a cluster, partitions, and other configurations suitable for implementing an embodiment.

Device 132 is an example of a device described herein. For example, device 132 can take the form of a smartphone, a tablet computer, a laptop computer, client 110 in a stationary or a portable form, a wearable computing device, or any other suitable device. Device 132 includes a client application 134 configured to perform functions of client application 134 described herein. Any software application described as executing in another data processing system in FIG. 1 can be configured to execute in device 132 in a similar manner. Any data or information stored or produced in another data processing system in FIG. 1 can be configured to be stored or produced in device 132 in a similar manner.

Health monitoring device 136 is configured to monitor health status information associated with a user such as exercise habits and heart rate measurements, and send the health status information to a server such as server 104. For example, health monitoring device 136 can take the form of a wearable fitness monitor or a smart watch.

Application 105 implements an embodiment described herein. In other embodiments, application 105 may be configured to perform the sub-optimal health detection and alert generation functions described herein. Database(s) 109, such as a user profile database, an image database, and/or a feature/symptom correlation database may be stored in storage 108 as shown or supplied by another source (not shown).

Servers 104 and 106, storage unit 108, and clients 110, 112, and 114, and device 132 may couple to network 102 using wired connections, wireless communication protocols, or other suitable data connectivity. Clients 110, 112, and 114 may be, for example, personal computers or network computers.

In the depicted example, server 104 may provide data, such as boot files, operating system images, and applications to clients 110, 112, and 114. Clients 110, 112, and 114 may be clients to server 104 in this example. Clients 110, 112, 114, or some combination thereof, may include their own data, boot files, operating system images, and applications. Data processing environment 100 may include additional servers, clients, and other devices that are not shown.

In the depicted example, data processing environment 100 may be the Internet. Network 102 may represent a collection of networks and gateways that use the Transmission Control Protocol/Internet Protocol (TCP/IP) and other protocols to communicate with one another. At the heart of the Internet is a backbone of data communication links between major nodes or host computers, including thousands of commercial, governmental, educational, and other computer systems that route data and messages. Of course, data processing environment 100 also may be implemented as a number of different types of networks, such as for example, an intranet, a local area network (LAN), or a wide area network (WAN). FIG. 1 is intended as an example, and not as an architectural limitation for the different illustrative embodiments.

Among other uses, data processing environment 100 may be used for implementing a client-server environment in which the illustrative embodiments may be implemented. A client-server environment enables software applications and data to be distributed across a network such that an application functions by using the interactivity between a client data processing system and a server data processing system. Data processing environment 100 may also employ a service oriented architecture where interoperable software components distributed across a network may be packaged together as coherent business applications. Data processing environment 100 may also take the form of a cloud, and employ a cloud computing model of service delivery for enabling convenient, on-demand network access to a shared pool of configurable computing resources (e.g. networks, network bandwidth, servers, processing, memory, storage, applications, virtual machines, and services) that can be rapidly provisioned and released with minimal management effort or interaction with a provider of the service.

With reference to FIG. 2, this figure depicts a block diagram of a data processing system in which illustrative embodiments may be implemented. Data processing system 200 is an example of a computer, such as servers 104 and 106, or clients 110, 112, and 114 in FIG. 1, or another type of device in which computer usable program code or instructions implementing the processes may be located for the illustrative embodiments.

Data processing system 200 is also representative of a data processing system or a configuration therein, such as data processing system 132 in FIG. 1 in which computer usable program code or instructions implementing the processes of the illustrative embodiments may be located. Data processing system 200 is described as a computer only as an example, without being limited thereto. Implementations in the form of other devices, such as device 132 in FIG. 1, may modify data processing system 200, such as by adding a touch interface, and even eliminate certain depicted components from data processing system 200 without departing from the general description of the operations and functions of data processing system 200 described herein.

In the depicted example, data processing system 200 employs a hub architecture including North Bridge and memory controller hub (NB/MCH) 202 and South Bridge and input/output (I/O) controller hub (SB/ICH) 204. Processing unit 206, main memory 208, and graphics processor 210 are coupled to North Bridge and memory controller hub (NB/MCH) 202. Processing unit 206 may contain one or more processors and may be implemented using one or more heterogeneous processor systems. Processing unit 206 may be a multi-core processor. Graphics processor 210 may be coupled to NB/MCH 202 through an accelerated graphics port (AGP) in certain implementations.

In the depicted example, local area network (LAN) adapter 212 is coupled to South Bridge and I/O controller hub (SB/ICH) 204. Audio adapter 216, keyboard and mouse adapter 220, modem 222, read only memory (ROM) 224, universal serial bus (USB) and other ports 232, and PCI/PCIe devices 234 are coupled to South Bridge and I/O controller hub 204 through bus 238. Hard disk drive (HDD) or solid-state drive (SSD) 226 and CD-ROM 230 are coupled to South Bridge and I/O controller hub 204 through bus 240. PCI/PCIe devices 234 may include, for example, Ethernet adapters, add-in cards, and PC cards for notebook computers. PCI uses a card bus controller, while PCIe does not. ROM 224 may be, for example, a flash binary input/output system (BIOS). Hard disk drive 226 and CD-ROM 230 may use, for example, an integrated drive electronics (IDE), serial advanced technology attachment (SATA) interface, or variants such as external-SATA (eSATA) and micro- SATA (mSATA). A super I/O (SIO) device 236 may be coupled to South Bridge and I/O controller hub (SB/ICH) 204 through bus 238.

Memories, such as main memory 208, ROM 224, or flash memory (not shown), are some examples of computer usable storage devices. Hard disk drive or solid state drive 226, CD-ROM 230, and other similarly usable devices are some examples of computer usable storage devices including a computer usable storage medium.

An operating system runs on processing unit 206. The operating system coordinates and provides control of various components within data processing system 200 in FIG. 2. The operating system may be a commercially available operating system for any type of computing platform, including but not limited to server systems, personal computers, and mobile devices. An object oriented or other type of programming system may operate in conjunction with the operating system and provide calls to the operating system from programs or applications executing on data processing system 200.

Instructions for the operating system, the object-oriented programming system, and applications or programs, such as application 105 in FIG. 1, are located on storage devices, such as in the form of code 226A on hard disk drive 226, and may be loaded into at least one of one or more memories, such as main memory 208, for execution by processing unit 206. The processes of the illustrative embodiments may be performed by processing unit 206 using computer implemented instructions, which may be located in a memory, such as, for example, main memory 208, read only memory 224, or in one or more peripheral devices.

Furthermore, in one case, code 226A may be downloaded over network 201A from remote system 201B, where similar code 201C is stored on a storage device 201D. In another case, code 226A may be downloaded over network 201A to remote system 201B, where downloaded code 201C is stored on a storage device 201D.

The hardware in FIGS. 1-2 may vary depending on the implementation. Other internal hardware or peripheral devices, such as flash memory, equivalent non-volatile memory, or optical disk drives and the like, may be used in addition to or in place of the hardware depicted in FIGS. 1-2. In addition, the processes of the illustrative embodiments may be applied to a multiprocessor data processing system.

In some illustrative examples, data processing system 200 may be a personal digital assistant (PDA), which is generally configured with flash memory to provide non-volatile memory for storing operating system files and/or user-generated data. A bus system may comprise one or more buses, such as a system bus, an I/O bus, and a PCI bus. Of course, the bus system may be implemented using any type of communications fabric or architecture that provides for a transfer of data between different components or devices attached to the fabric or architecture.

A communications unit may include one or more devices used to transmit and receive data, such as a modem or a network adapter. A memory may be, for example, main memory 208 or a cache, such as the cache found in North Bridge and memory controller hub 202. A processing unit may include one or more processors or CPUs.

The depicted examples in FIGS. 1-2 and above-described examples are not meant to imply architectural limitations. For example, data processing system 200 also may be a tablet computer, laptop computer, or telephone device in addition to taking the form of a mobile or wearable device.

Where a computer or data processing system is described as a virtual machine, a virtual device, or a virtual component, the virtual machine, virtual device, or the virtual component operates in the manner of data processing system 200 using virtualized manifestation of some or all components depicted in data processing system 200. For example, in a virtual machine, virtual device, or virtual component, processing unit 206 is manifested as a virtualized instance of all or some number of hardware processing units 206 available in a host data processing system, main memory 208 is manifested as a virtualized instance of all or some portion of main memory 208 that may be available in the host data processing system, and disk 226 is manifested as a virtualized instance of all or some portion of disk 226 that may be available in the host data processing system. The host data processing system in such cases is represented by data processing system 200.

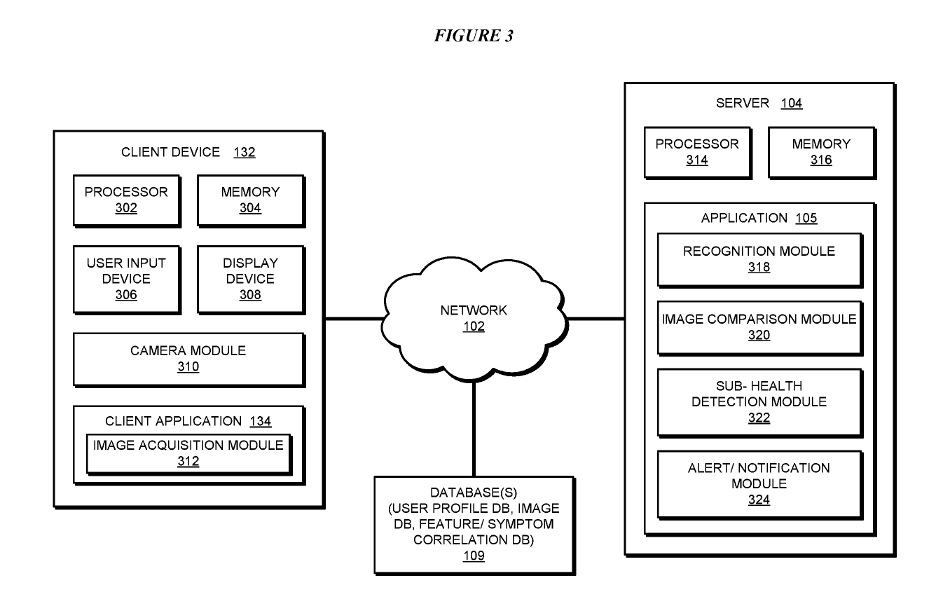

With reference to FIG. 3, this figure depicts a block diagram of an example configuration for sub-optimal health detection and alert generation using a time series of images of a user in accordance with an illustrative embodiment. The example embodiment includes client device 132, server 104, and database(s) 109 in communication via network 102.

Client device 132 is an example of device 132 of FIG. 1 and includes a processor 302, a memory 304, a user input device 306, a display device 308, a camera module 310, and client application 134. Processor 302 is configured to retrieve instructions from memory 304 and execute the instructions to perform various operations of client device 132 as described herein. In one or more embodiments, user input device 306 is configured to provide one or more input devices to allow the user to interact with client device 132. In one or more embodiments, display device 308 is configured to display information such as health status information to a user of client device 132. Camera module 310 is configured to capture or more images such as images of user of client device 132. Client application 134 includes an image acquisition module 312 configured to capture a series of images of a user over a period of time using camera module 310.

Server 104 is an example of server 104 of FIG. 1 and includes a processor 314, a memory 316, and application 105. Processor 314 is configured to retrieve instructions from memory 316 and execute the instructions to perform various operations of server 104 as described herein. Application 105 includes a recognition module 318, an image comparison module 320, a sub-health detection module 322, and an alert/notification module 324. Application 105 is configured to perform the operations associated with sub-optimal health detection and alert generation as described herein. Recognition module 318 is configured to recognize a user's face or other body portion and extract features that are related to a health status of the user. Image comparison module 320 is configured to compare a series of images against one another across an adjustable time window. Sub-health detection module 322 is configured to receive input from image comparison module 320 and a knowledge of mapping between facial and/or body feature changes and sub-optimal health status such as internal health or skin disease, and detect any anomaly that may be indicative of sub-optimal health status of the user. Alert/notification module 324 is configured to generate and send alerts or notifications indicative of a detected long-term or short-term health status change to the user and/or a physician or other suitable entity.

Database(s) 109 include one or more databases such as a user profile database including a profile of one or more users, an image database including one or more images of the user captured over a period of time, and a feature/symptom correlation database that includes information correlating a specific facial or body feature to a particular symptom indicative of a health status of a user.

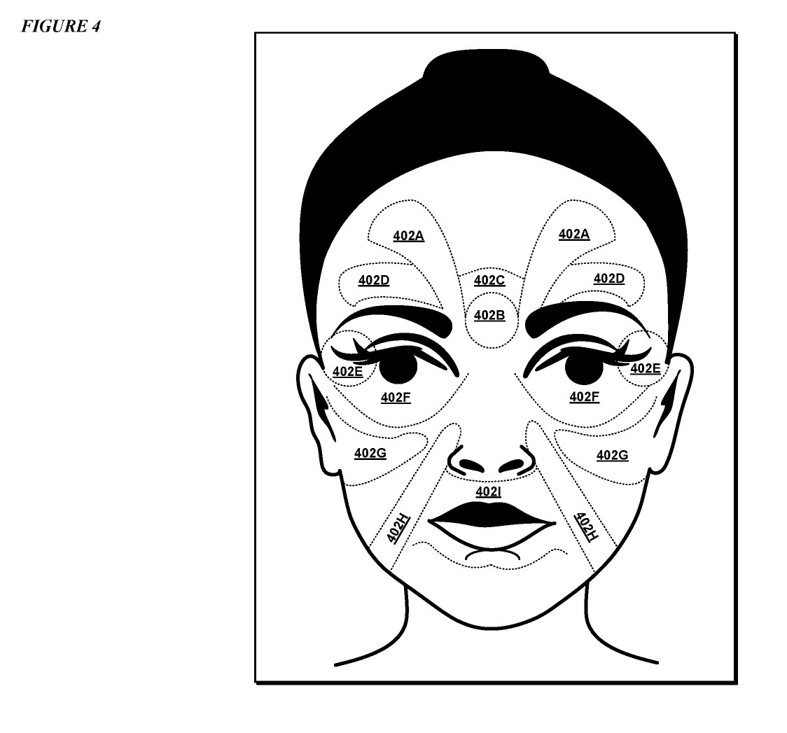

With reference to FIG. 4, this figure depicts example facial characteristics in accordance with an illustrative embodiment. The illustration of FIG. 4 depicts a number of facial characteristics 402A-402I on portions of a user in which a current appearance or change in appearance over a time period of one or more facial characteristics 402A-402I is indicative of a particular health condition of the user. For example, a change appearance in one or more of facial characteristics 402A-402I may be indicative of a problem with a particular organ such as a small intestine, bladder, liver, heart, kidneys, stomach, lungs, large intestine, or other medical problems of the user.

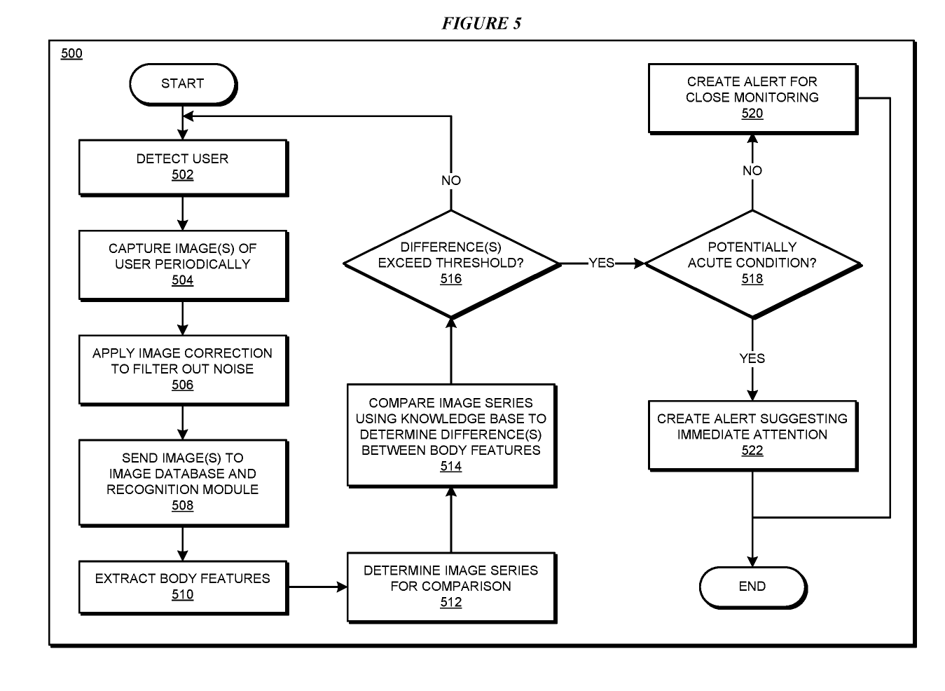

With reference to FIG. 5, this figure depicts a flowchart of an example process 500 for sub-optimal health detection and alert generation using a time series of images of a user in accordance with an illustrative embodiment. In 502, client application 134 of client device 132 detects a user within view of a camera of client device 132. In 504, client device 132 captures one or more images of a face or other body part of the user over a period of time. In a particular embodiment, client device 132 periodically captures a time series of images of the user over a period of time. In 506, client device 132 applies image correction to the image(s) to filter out noise due to one or more environmental factors present during the capturing of the image(s) or conditions of the user during the capturing such as lighting conditions, weather, and the presence of the makeup on the user. In 508, client device 132 sends the image(s) to an image database of database 109 and/or recognition module 318 within application 107 of server 104.

In 510, recognition module 318 within application 107 of server 104 extracts one or more facial features (or other body features) from the image(s) after receiving the image(s) in which the extracted features are features in which a change in appearance of the feature over a period of time may be indicative of a sub-optimal health condition of the user. In particular embodiments, server 104 receives the image(s) from either database 109 or directly from client device 132. In 512, image comparison module 320 determines a time series of images previously captured over a predetermined period of time to compare to the most recently received image. In 514, image comparison module 320 compares the image series using a knowledge base to determine differences between one or more facial features (or other body features) that are indicative of a sub-optimal health condition of the user. In a particular embodiment, the knowledge bases includes one or more references images that correlate a specific facial or body feature to a particular potential health condition. In 516, image sub-health detection module 322 determines whether a difference in the facial or body features in the series of images exceed a predetermined threshold value. If the difference does not exceed the threshold value, process 500 returns to 502. If the difference does exceed the threshold value, process 500 continues to 518.

In 518, sub-health detection module 322 determines whether the detected health condition is a potentially acute condition. A potentially acute condition is a condition that may require prompt or immediate medical attention. If sub-health detection module 322 determines that the detected health condition is not a potentially acute condition, process 500 continue to 520. In 520, alert/notification module 324 creates an alert for close monitoring of the health condition and sends the alert to the user. In particular embodiments, close monitoring of the health condition may include suggesting to the user to schedule an appointment with a health care provider to have a medical professional evaluate the potential health condition. If sub-health detection module 322 determines that the detected health condition is a potentially acute condition, process 500 continue to 522. In 522, alert/notification module 324 creates an alert suggesting immediate attention to the potential health condition and sends the alert to the user. The user may then contact a health provider, such as an emergency room, for immediate attention to the potential health condition.

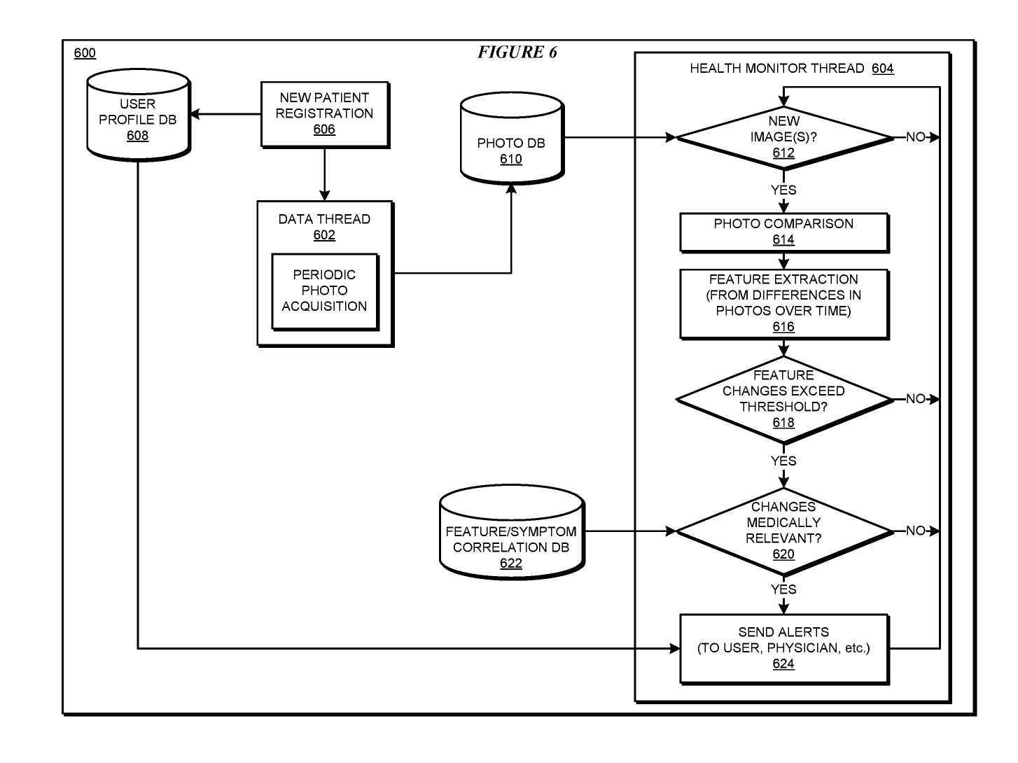

With reference to FIG. 6, this figure depicts a flowchart of another example process 600 for sub-optimal health detection and alert generation using a time series of images of a user in accordance with an illustrative embodiment. FIG. 6 illustrates a multi-threaded process 600 in which a data thread 602 runs on client device 132 and a health monitor thread 604 runs on server 104. Data thread 602 of client device 132 receives new patient registration information 606 from a user and stores the new patent information in a user profile associated with a user in a user profile database 608. In the embodiment, data thread 602 is configured to perform periodic photo acquisition of images of the user and store the images in photo database 610 as a time series of images.

Health monitor thread 604 running on server 104 determines whether health monitor thread 604 has received one or more new images from photo database 610 in 612. If no new image has been received by health monitor thread 604, process 600 remains at block 612. If a new image has been received by health monitor thread 604, process 600 proceeds to block 614. In 614, health monitor thread 604 performs a photo comparison to compare a time series of images of the user to determine one or more differences between the images in the series of images.

In 616, health monitor thread 604 performs feature extraction from the differences in the photos over time to identify feature changes that may be indicative of a sub-optimal health condition in the user. In 618, health monitor thread 604 determines whether the feature changes exceed a predetermined threshold. If the feature changes do not exceed a predetermined threshold, process 600 returns to 612. If the feature changes exceed the predetermined threshold, process 600 continues to 620. In 620, health monitor thread 604 determines whether the feature changes are medically relevant based upon matching the feature changes to symptoms correlated to the features from a feature/symptom correlation database 622. If the feature changes are determined to not be medically relevant, process 600 returns to 612. If the feature changes are determined to be medically relevant, process 600 continues to 624.

In 624, health monitor thread 604 sends one or more alerts notifying the user and/or another entity that a potentially medically relevant sub-optimal health condition may exist with the user. In a particular embodiment, health monitor thread 604 receives user profile information about the user from user profile database 608 including identifying information of the user and other entities such as the user's physician or person of contact to determine to whom the alerts should be sent.

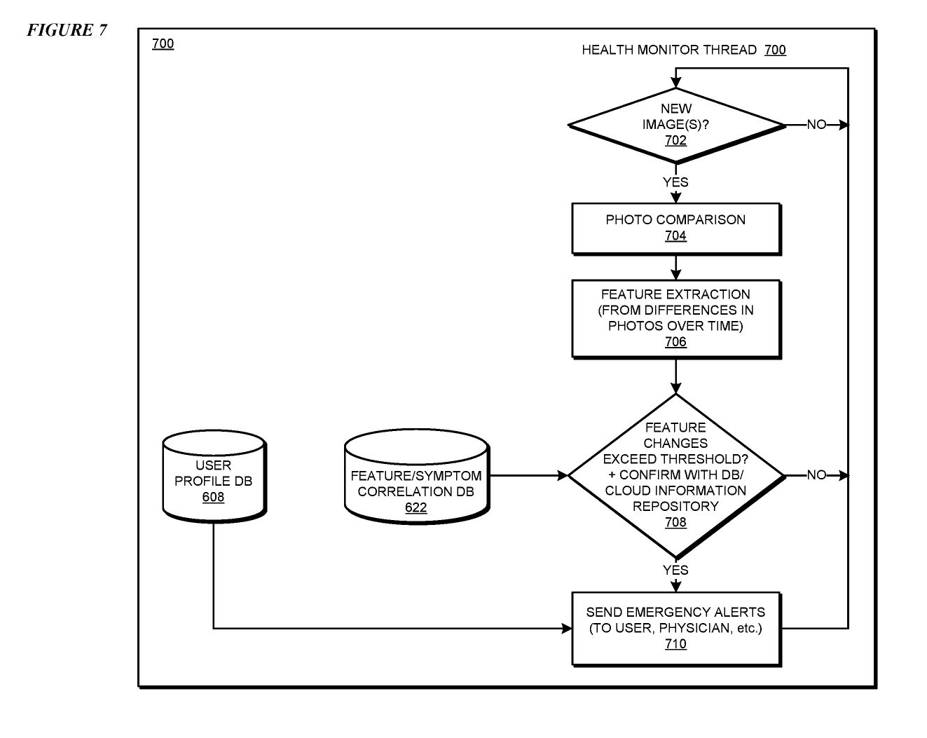

FIG. 7 depicts a flowchart of another example process 700 for sub-optimal health detection and alert generation using a time series of images of a user in accordance with an illustrative embodiment. In the embodiment depicted in FIG. 7, a health monitor thread 700 further includes a function for monitoring and detecting an emergency event from a images associated with a user and immediately alert the user if an emergency event is detected. In 702, health monitor thread 700 running on server 104 determines whether health monitor thread 700 has received one or more new images such as from photo database 610 of FIG. 6. If no new image has been received by health monitor thread 700, thread 700 remains at block 702. If a new image has been received by health monitor thread 604, thread 700 proceeds to block 704. In 704, health monitor thread 700 performs a photo comparison to compare a time series of images of the user to determine one or more differences between the images in the series of images.

In 706, health monitor thread 700 performs feature extraction from the differences in the photos over time to identify feature changes that may be indicative of a sub-optimal health condition in the user. In 708, health monitor thread 604 determines whether the feature changes exceed a second predetermined threshold greater than the first predetermined threshold (e.g., exceeds the threshold dramatically) based upon matching the feature changes to symptoms correlated to the features from feature/symptom correlation database 622. If the feature changes are determined to not exceed the second threshold, thread 700 returns to 702. If the feature changes exceed the second threshold, thread 700 continues to 710.

In 710, health monitor thread 700 sends one or more emergency alerts notifying the user and/or another entity that an emergency health condition may exist with the user and that the user should contact a medical provider immediately or as soon as possible. In a particular embodiment, health monitor thread 700 receives user profile information about the user from user profile database 608 including identifying information of the user and other entities such as the user's physician or an emergency contact to determine to whom the alerts should be sent.

Thus, a computer implemented method, system or apparatus, and computer program product are provided in the illustrative embodiments for providing sub-optimal health detection and alert generation using a time series of images of a user and other related features, functions, or operations. Where an embodiment or a portion thereof is described with respect to a type of device, the computer implemented method, system or apparatus, the computer program product, or a portion thereof, are adapted or configured for use with a suitable and comparable manifestation of that type of device.

Where an embodiment is described as implemented in an application, the delivery of the application in a Software as a Service (SaaS) model is contemplated within the scope of the illustrative embodiments. In a SaaS model, the capability of the application implementing an embodiment is provided to a user by executing the application in a cloud infrastructure. The user can access the application using a variety of client devices through a thin client interface such as a web browser (e.g., web-based e-mail), or other light-weight client-applications. The user does not manage or control the underlying cloud infrastructure including the network, servers, operating systems, or the storage of the cloud infrastructure. In some cases, the user may not even manage or control the capabilities of the SaaS application. In some other cases, the SaaS implementation of the application may permit a possible exception of limited user-specific application configuration settings.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

What is claimed is:

1. A method comprising:
receiving a series of images of a body part of a user, the series of images of the body part of the user captured over a predetermined time period;
extracting a feature of the body part from the series of images, in which a change in appearance of the extracted feature over the predetermined time period is indicative of a potential health condition of the user;
comparing the extracted feature to one or more reference images from a knowledge base to determine a difference between the extracted feature and the one or more reference images, the one or more reference images correlating a particular body feature to a particular potential health condition;
detecting a potential health condition of the user based upon the comparing; and
sending a notification based upon the detected potential health condition.

2. The method of claim 1, wherein detecting the potential health condition further comprises:
determining that the difference exceeds a threshold.

3. The method of claim 1, further comprising:
applying image correction to at least one of the series of images to filter out noise due to one or more environmental factors present during capturing of the at least one image.

4. The method of claim 1, wherein the notification is sent to at least one of the user and a medical provider associated with the user.

5. The method of claim 1, further comprising:
determining that the potential health condition is a potentially acute health condition.

6. The method of claim 5, wherein the notification is an emergency alert indicating the potentially acute health condition.

7. The method of claim 1, further comprising:
receiving activity information from a wearable device associated with the user, the activity information indicative of activity of the user; and
determining a reason for the detected potential health condition based upon the activity information.

8. The method of claim 1, wherein the body part comprises a face of the user, the feature including a facial feature.

9. The method of claim 1, wherein the body part comprises at least one of a face of the user, a hand of the user, a torso of the user, a leg of the user, a neck of the user, and a skin of the user.

10. The method of claim 1, wherein the feature includes at least one of a facial anomaly, a skin condition of the body part, an eye condition of the user, a teeth condition of the user, a change in shape of the body part, and a color of the body part.

11. A computer usable program product comprising one or more computer-readable storage devices, and program instructions stored on at least one of the one or more storage devices, the stored program instructions comprising:
program instructions to receive a series of images of a body part of a user, the series of images of the body part of the user captured over a predetermined time period;
program instructions to extracting a feature of the body part from the series of images, in which a change in appearance of the extracted feature over the predetermined time period is indicative of a potential health condition of the user;
program instructions to compare the extracted feature to one or more reference images from a knowledge base to determine a difference between the extracted feature and the one or more reference images, the one or more reference images correlating a particular body feature to a particular potential health condition;
program instructions to detect a potential health condition of the user based upon the comparing; and
program instructions to send a notification based upon the detected potential health condition.

12. The computer usable program product of claim 11, wherein the program instructions to detect the potential health condition further include program instructions to determine that the difference exceeds a threshold.

13. The computer usable program product of claim 11, further comprising:
program instructions to apply image correction to at least one of the series of images to filter out noise due to one or more environmental factors present during capturing of the at least one image.

14. The computer usable program product of claim 11, wherein the notification is sent to at least one of the user and a medical provider associated with the user.

15. The computer usable program product of claim 11, further comprising program instructions to determine that the potential health condition is a potentially acute health condition.

16. The computer usable program product of claim 15, wherein the notification is an emergency alert indicating the potentially acute health condition.

17. The computer usable program product of claim 11, further comprising:
program instructions to receive activity information from a wearable device associated with the user, the activity information indicative of activity of the user; and
program instructions to determine a reason for the detected potential health condition based upon the activity information.

18. The computer usable program product of claim 11, wherein the body part comprises at least one of a face of the user, a hand of the user, a torso of the user, a leg of the user, a neck of the user, and a skin of the user.

19. A computer system comprising one or more processors, one or more computer-readable memories, and one or more computer-readable storage devices, and program instructions stored on at least one of the one or more storage devices for execution by at least one of the one or more processors via at least one of the one or more memories, the stored program instructions comprising:
program instructions to receive a series of images of a body part of a user, the series of images of the body part of the user captured over a predetermined time period;
program instructions to extracting a feature of the body part from the series of images, in which a change in appearance of the extracted feature over the predetermined time period is indicative of a potential health condition of the user;
program instructions to compare the extracted feature to one or more reference images from a knowledge base to determine a difference between the extracted feature and the one or more reference images, the one or more reference images correlating a particular facial feature to a particular potential health condition;
program instructions to detect a potential health condition of the user based upon the comparing; and
program instructions to send a notification based upon the detected potential health condition.

20. The computer system of claim 19, wherein the program instructions to detect the potential health condition further include program instructions to determine that the difference exceeds a threshold.
